(19) United States
(12) Patent Application Publication
(10) Pub. No.: US 2005/0228464 A1
(43) Pub. Date: Oct. 13, 2005

(54) REWARMER

(76) Inventor: David Jackie Hammac, Norwood, NY (US)

Correspondence Address: RICHARD D. FUERLE, 1711 W. RIVER RD., GRAND ISLAND, NY, 14072, US

(21) Appl. No.: 10/709,070

(22) Filed: Apr. 10, 2004

(51) Int. Cl. A61F 7/00

(52) U.S. Cl. 607/108; 607/112

(57) ABSTRACT

A rewarmer is made from a flexible material that can be laid flat. The material has a central portion that covers the back of a person lying supine on it, at least one left flap that can be brought over the left side of the person, at least one right flap that can be brought over the right side of the person, and a way to hold the left flap and the right flap together. There is an electrical resistance heating element within the flexible material and the electrical resistance heating element can be connected to a source of electricity. To use the rewarmer to treat a person who is suffering from hypothermia, the rewarmer is laid flat on a substantially horizontal surface. The person is placed supine on the rewarmer, so that at least one left flap is on his left side and at least one right flap is on his right side. The left flaps are attached to the right flaps over the torso of the person and the electrical resistance heating element is connected to a source of electricity.

### BACKGROUND OF INVENTION

This invention relates to an electrically heated garment that can be wrapped around a person who has hypothermia to increase his body temperature. In particular, it relates to a garment that can be laid flat and contains an electrical resistance heating element, so that when a hypothermic person lies supine on the garment, flaps can be brought over his torso and attached.

Hypothermia is a condition where a person's body temperature falls below normal, usually due exposure to low temperatures. For example, a person may become lost, injured, or otherwise incapacitated outdoors during winter or at night when the temperature falls, and be unprepared for the cold. The body temperature of a person who falls through thin ice, has a boating accident, or otherwise ends up in cold water will quickly reach a dangerously low temperature. Untreated, hypothermia can result in frostbite, cardiac arrest, coma, and death.

Present treatments include providing warm humid air for the person to breathe and rapid core rewarming. However, heat-generating equipment may not be available, especially if the person is in an isolated area. Also, if the equipment is costly, heavy, or bulky many rescue units may not have it or be able to transport it to where it is needed.

A number of torso warmers have been made, but these are primarily for healthy people who simply want to keep warm in cold weather. These warmers are made to be used by people who are not injured and are capable of putting them on in a standing position, as one would put on a vest, jacket, or coat. They usually have openings through which the arms must be inserted, which may not be easy to do when a person is injured, lying down, or cannot move. Examples of torso warmers can be found in U.S. Pat. Nos. 6,439,942; 3,999,037; 6,049,062; 5,977,517; and 5,893,991.

### SUMMARY OF INVENTION

The rewarmer of this invention is suitable for warming hypothermic people who may be injured, are lying down, cannot move, or cannot be safely moved. The rewarmer can be laid flat and a person can simply lie on it or be placed upon it. Flaps are attached over him, an electrical connection is made and heat is quickly generated around his torso, head, neck, and buttocks. There are no arm openings or legs openings through which a person”s arms or legs must be inserted, so that even a person who is injured can be warmed without moving him, other than to slide or roll him on the rewarmer.

The rewarmer warms the torso first, rather than the limbs, so that the torso can accept colder blood coming from the limbs without suddenly cooling (known as core after-drop). The rewarmer can be heated prior to reaching a hypothermic person and heating can continue even after the person is hospitalized, by shifting from battery power to 110 VAC power.

The rewarmer is lightweight, takes up little space, and can be taken anywhere, even where vehicles cannot go. It can be battery powered, so that it need not be used near a vehicle or a 110 VAC line.

### BRIEF DESCRIPTION OF DRAWINGS

FIG. 1 is a plan view of a certain presently preferred embodiment of a rewarmer according to this invention.

FIG. 2 is a plan view of the rewarmer of FIG. 1 showing a person lying supine upon it.

FIG. 3 is a plan view of the rewarmer of FIGS. 1 and 2, showing the rewarmer covering the chest and abdomen of a person lying supine upon it.

FIG. 4 is a plan view of an alternative embodiment of a rewarmer according to this invention, partially cut-away to show a portion of the electrical resistance wiring.

FIG. 5 is a plan view of the rewarmer of FIG. 4, showing the rewarmer covering the abdomen and the right side of the chest of a person lying supine upon it.

FIG. 6 is a side view in section through a rewarmer according to this invention.

### DETAILED DESCRIPTION

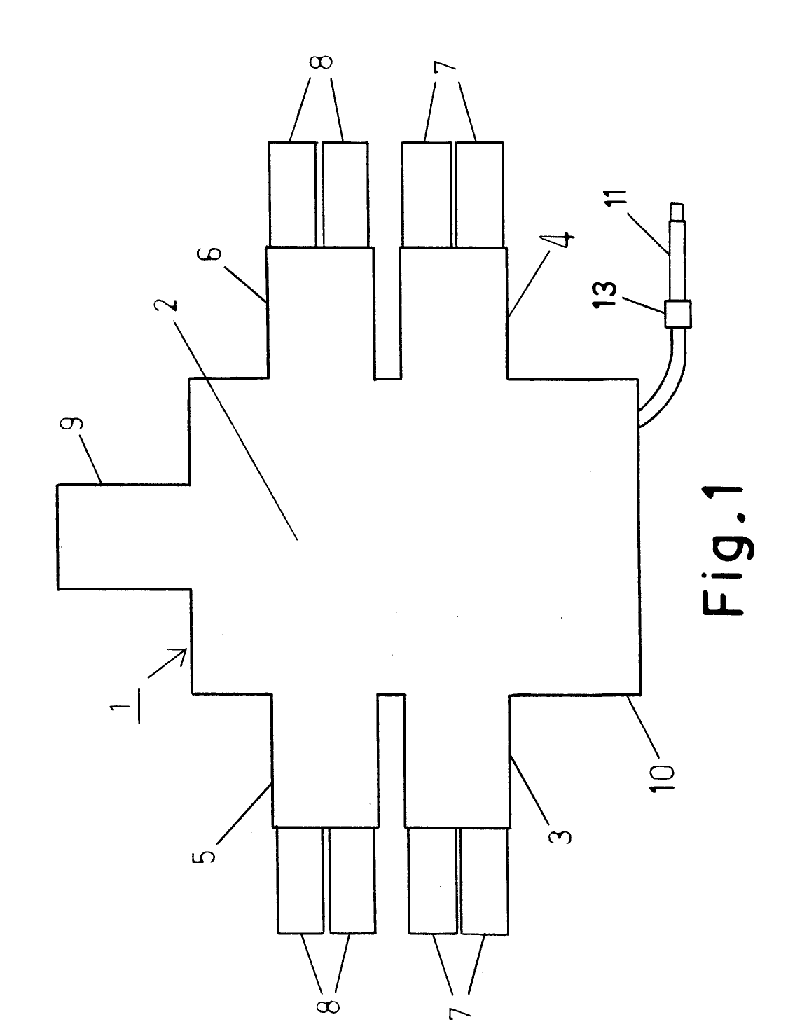

In FIG. 1, rewarmer 1, according to this invention, is laid out flat on a surface that is horizontal, or as horizontal as possible under the circumstances. The surface may be the ground, a stretcher, a bench, a table, the floor, or another more or less flat surface. Rewarmer 1 has a central portion 2 that is large enough to cover the torso of a person placed upon it. Attached to central portion 2 is a right abdominal flap 3 and an opposing left abdominal flap 4. Also attached to central portion 2 is a right chest flap 5 and an opposing left chest flap 6.

At the end of each of the abdominal flaps 3 and 4 and each of the chest flaps 5 and 6 are attached two straps 7 and 8, respectively. Preferably, the straps on one side have a multiplicity of small hooks on the surface while the straps on the opposing side have fabric on the surface, so that the hooks can engage the fabric and hold the opposing straps releaseably together, a product sold as Velcro. Alternatively, the straps on one side could have a buckle at the end and the straps on the other side could have apertures therethrough, so that the opposing straps can be attached by buckling them together. The opposing straps could also be held together by snaps, buttons, or other means or they could be simply tied together. Preferably, the straps contain elastic so that they are stretchable, thereby ensuring a snug fit of the flaps around a person's body.

The use of separate chest and abdominal flaps helps the rewarmer to fit snugly around people, even though they may have a variety of different body shapes. Also, if a person needs to be defibrillated, the chest flaps can be undone while the abdominal flaps continue to warm the body. Preferably, for an adult size rewarmer, the chest flaps should be attachable to enclose a circumference between about 30 to about 45 inches and the abdominal flaps should be attachable to enclose a circumference of about 30 to about 60 inches. The flaps on a child size rewarmer would, of course, enclose smaller chest and abdominal circumferences.

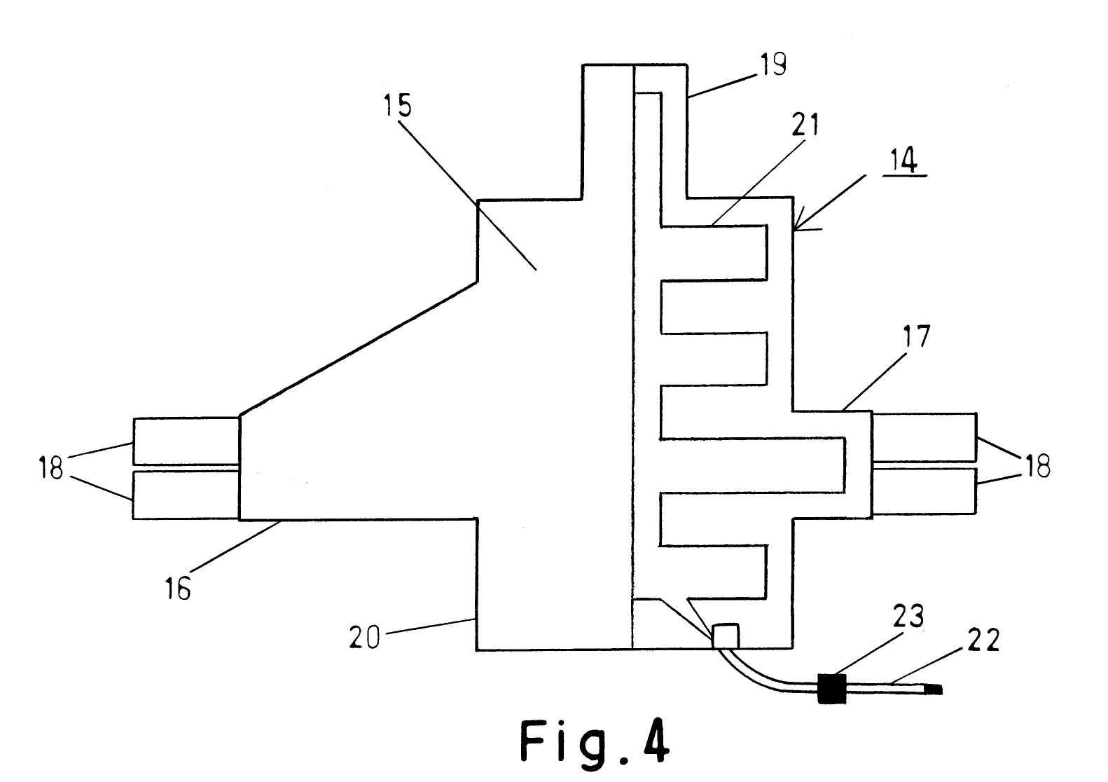

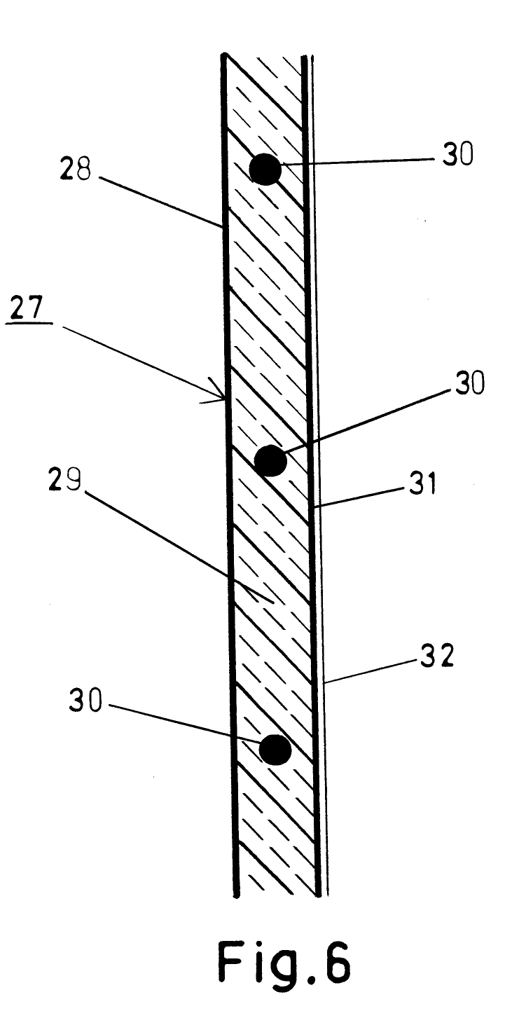

Preferably, a head flap 9 is attached to the top of central portion 2 in the middle to protect a person's head and provide heat to the head and neck of a person. Also, a buttocks flap 10 is preferably attached to the base of central portion 2 in the middle to protect a person's buttocks and provide heat thereto. A cord 11 is provided to connect the electrical resistance element within rewarmer 1 (see FIGS. 4 and 6) to a source of electricity.

The dimensions of the rewarmer may be selected to correspond to the size of the majority of people in the area where it is to be used. A length from the end of the head flap to the end of the buttocks flap of about 3 to about 3½ feet may be suitable for most adults and a length of about 2 to about 2½ feet for many children.

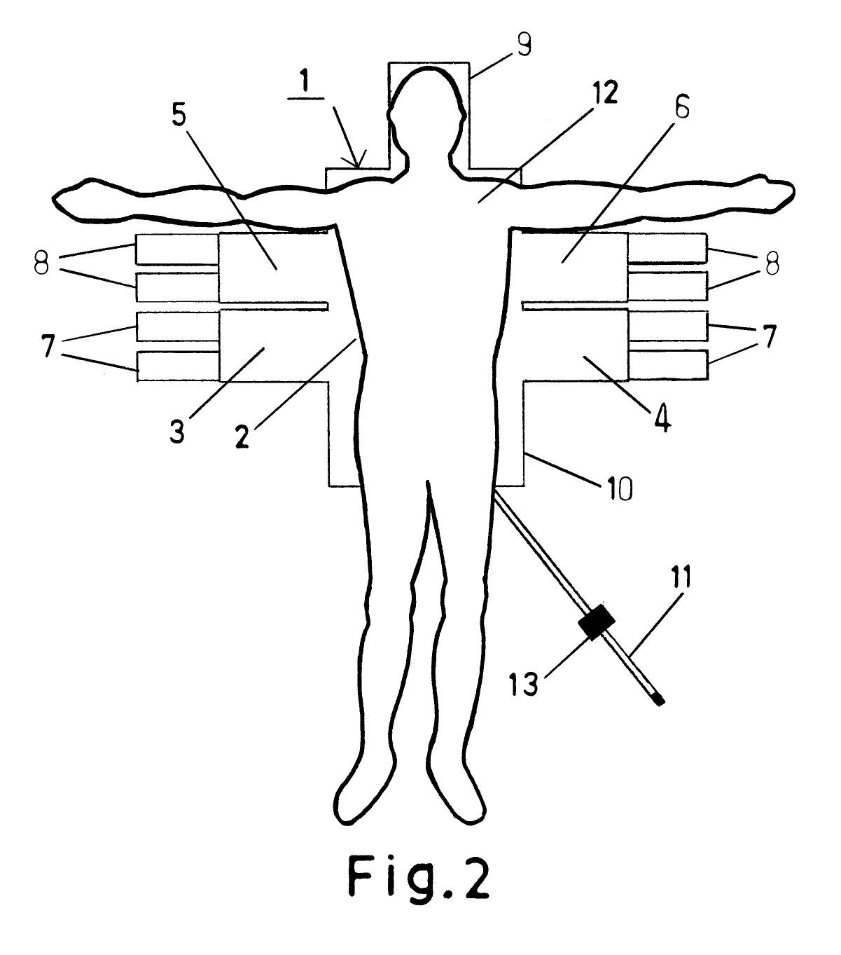

FIG. 2 shows the position of a person 12 lying supine on rewarmer 1. A person who is suffering from hypothermia is carried, rolled, or places himself in the position shown in FIG. 2 in order to receive heat from rewarmer 2.

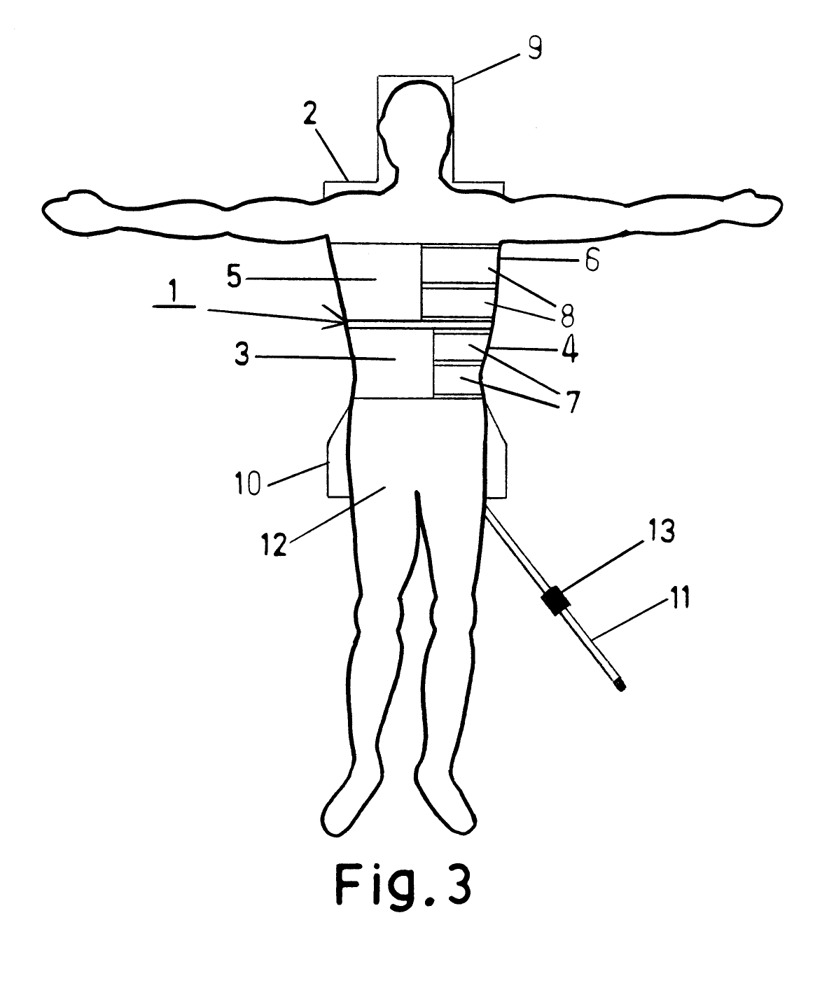

In FIG. 3, opposing chest and abdominal straps 7 and 8, respectively, of rewarmer 1 have been attached, thereby enclosing the torso of person 12 within rewarmer 1. The person's arms and legs extend from rewarmer 1 and are not confined. Rewarmer 1 is then activated, by connecting cord 11 to a source of electricity. Cord 11 is preferably provided with a thermostat 13 which controls the flow of electricity into the electrical resistance element, so that the current can be shut off or increased to a maximum as needed, thereby controlling the temperature of rewarmer 1. For example, in a remote location a low power setting might be used to conserve the batteries. A high power setting might be used when ample power is available or when it is necessary to raise a person's temperature quickly.

In FIG. 4, an alternative rewarmer 14 has a central portion 15 to which is attached right abdominal flap 16 and left abdominal flap 17. Left abdominal flap 17 is rectangular in shape, but right abdominal flap 16 extends diagonally from the top right to the abdomen so that it covers the right chest of a person in addition to his abdomen, but leaves his left chest exposed for defibrillation. A pair of straps 18, similar to straps 7 and 8, are attached to the end of flaps 16 and 17. A head flap 19 and a buttocks flap 20 are also attached to central portion 15.

Rewarmer 14 is partially cut-away to show a portion of an electrical resistance heating element 21, which extends throughout rewarmer 14. In the rewarmers of this invention, the electrical resistance wire preferably passes through and heats not only the central portion, but also the abdominal, chest, head, and buttocks flaps as well. Element 21 forms a complete circuit and connecting cord 22 to a source of electricity permits electricity to pass through that circuit. The electricity is converted into heat, which quickly heats rewarmer 14. Cord 22 is preferably provided with a thermostat 23.

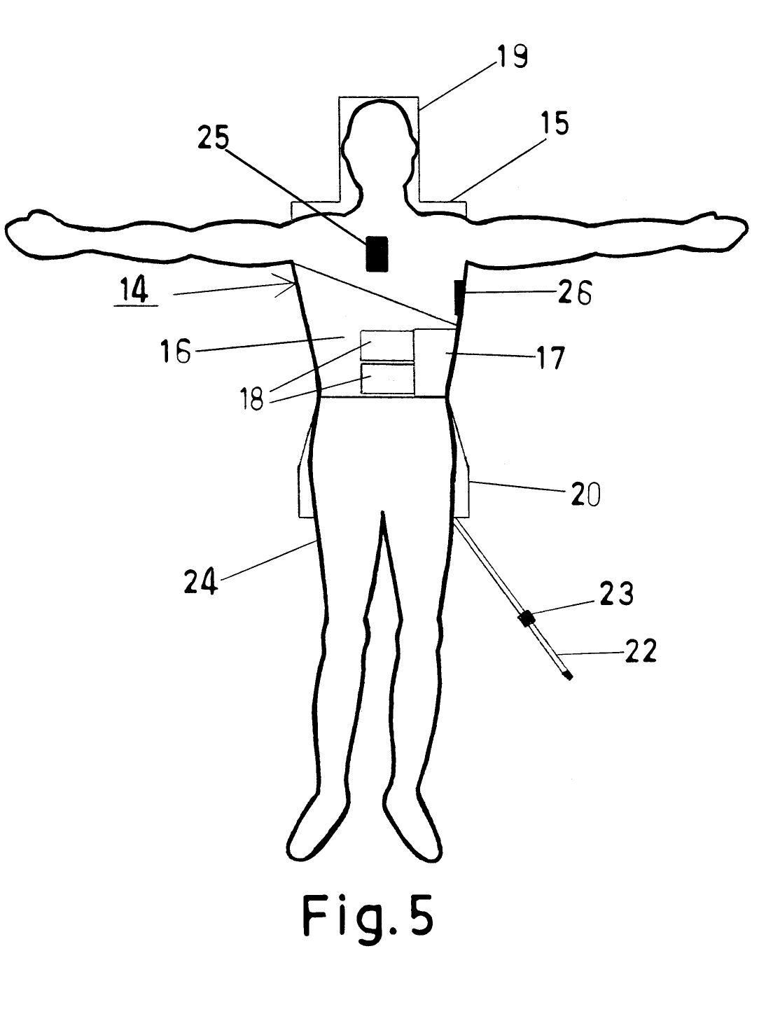

FIG. 5 shows straps 18 of rewarmer 14 attached over a person 24. The person's left chest is not covered by rewarmer 14, so that paddle placement areas 25 and 26 are exposed. Thus, if necessary, a defibrillator can be attached to person 24 and he can be treated without removing rewarmer 14.

FIG. 6 shows a preferred internal construction for a rewarmer 27 according to this invention. Outer layer 28 of rewarmer 27 is preferably made of a flexible, waterproof material that is electrically non-conductive (in case a defibrillator is used), durable, and easily cleaned. Examples of suitable materials include rubberized fiberglass reinforced material (sold as Zodiac for boat covers), polyurethane, polyethylene, synthetic rubber, vinyl, or another type of flexible plastic. Material 29 inside rewarmer 27 is preferably thermal insulation, such as cotton, polyamide (Nylon), down, polyurethane foam, wool, or fleece. Insulated electrical resistance heating element 30 passes throughout material 29. Element 30 is typically made of thin, flexible wires, but other materials could also be used. Element 30 is preferably used with 12 VDC as that is the standard used for vehicle batteries, portable batteries, and converters from 110 VAC. A flexible, waterproof liner 31 covers the inside of rewarmer 27. Preferably, a disposable liner 32 is removably attached to the inside of rewarmer 27 to protect rewarmer 27 from fluids from the person it is covering. Disposable liner 32 can be waterproof on the outside and absorbent on the inside, the side that is against the patient, so that it is comfortable.

The source of electricity for the rewarmer can be a battery, household current (110 VAC), or other source. A battery, preferably 12 VDC, is preferred so that the rewarmer can be used anywhere. A battery can be carried along with the rewarmer or the rewarmer can be plugged into the cigarette lighter of a vehicle so that the vehicle's battery can be used. If a 110 VAC current is used, the current is preferably rectified to DC, preferably 12 VDC.

1. A rewarmer comprising
(A) a flexible material that can be laid flat, said material having
(1) a central portion that covers the back of a person lying supine thereon;
(2) at least one left flap that can be brought over the left side of a person lying supine on said central portion;
(3) at least one right flap that can be brought over the right side of a person lying supine on said central portion;
(4) means for holding said left flap and said right flap together;
(B) an electrical resistance heating element within said flexible material; and
(C) means for connecting said electrical resistance heating element to a source of electricity.

2. A rewarmer according to claim 1 wherein a first pair of opposing flaps attach over the chest of said person and a second pair of opposing flaps attach over the abdomen of said person.

3. A rewarmer according to claim 2 wherein said opposing flaps that attach over the chest can accommodate a chest having a circumference of about 30 to about 45 inches and said opposing flaps that attach over the abdomen can accommodate an abdomen having a circumference of about 30 to about 60 inches.

4. A rewarmer according to claim 1 wherein said at least one right flap is a single flap that covers said person's abdomen and the right side of said person's chest and said at least one left flap is a single flap that covers said persons abdomen.

5. A rewarmer according to claim 1 wherein said flaps are attached together by means of a straps extending from said flaps, each strap having an array of small hooks that engage fabric on the opposing flap.

6. A rewarmer according to claim 1 wherein said flaps are attached by means of elastic flaps.

7. A rewarmer according to claim 1 wherein a flap that lies under said person's buttocks is attached to said central portion.

8. A rewarmer according to claim 1 wherein a flap that lies under said person's head and neck is attached to said central portion.

9. A rewarmer according to claim 1 wherein said source of electricity is a 12 volt battery.

10. A rewarmer according to claim 1 wherein said source of electricity 110 VAC that is converted to 12 VDC.

11. A rewarmer according to claim 1 including a thermostat for controlling the flow of said electricity through said electrical resistance heating element.

12. A rewarmer according to claim 1 wherein the outside of said rewarmer is waterproof.

13. A rewarmer according to claim 1 including a removable liner next to said person.

14. A method of rewarming a person who is suffering from hypothermia comprising
(A) laying a rewarmer according to claim 1 flat on a substantially horizontal surface;
(B) placing said person supine on said rewarmer, so that said at least one left flap is on the left side of his torso and said at least one right flap is on the right side of his torso;
(C) attaching said at least one left flap to said at least one right flap over the torso of said person; and
(D) connecting said electrical resistance heating element to a source of electricity.

15. A rewarmer comprising
(A) a flexible material that has no openings for arms and can be laid flat, said material having
(1) a central portion that covers the back of a person lying supine thereon;
(2) a single left flap fixed to said central portion that can be brought over the left side of said person's s abdomen;
(3) a single right flap fixed to said central portion that can be brought over the right side of said person's abdomen and the right side of said person's chest, but which leaves the left side of said person's chest exposed;
(4) means for attaching said left flap to said right flap;
(B) an electrical resistance heating element within said flexible material; and
(C) means for connecting said electrical resistance heating element to a source of electricity that is external to said rewarmer.

16. A rewarmer according to claim 15 wherein said left flap is attached to said right flap by means of straps that extend from said flaps, where each strap on one side has an array of small hooks that engage fabric on the strap on the opposite side.

17. A rewarmer according to claim 15 wherein a buttocks flap is fixed to said central portion and covers said person's s buttocks and a head flap is fixed to said central portion and covers said person's head and neck.

18. A rewarmer comprising
(A) a flexible material that has no openings for arms and can be laid flat, said material having
(1) a central portion that covers the back of a person lying supine thereon;
(2) two left flaps fixed to said central portion, one of which can be brought over said person to cover the left side of his chest and the other of which can be brought over said person to cover the left side of his abdomen;
(3) two right flaps fixed to said central portion, one of which can be brought over said person to cover the right side of his chest and the other of which can be brought over said person to cover the right side of his abdomen;
(4) means for attaching each left flap to an opposing right flap;
(B) an electrical resistance heating element within said flexible material; and
(C) means for connecting said electrical resistance heating element to a source of electricity that is external to said rewarmer.

19. A rewarmer according to claim 18 wherein said two left flaps are attached to said two right flaps by means of straps that extend from said flaps, where each strap on one side has an array of small hooks that engage fabric on the strap on the opposite side.

20. A rewarmer according to claim 18 wherein a buttocks flap is fixed to said central portion and covers said person's buttocks and a head flap is fixed to said central portion and covers said person's head and neck.
